(19) United States
(12) Patent Application Publication
(10) Pub. No.: US 2021/0267190 A1
(43) Pub. Date: Sep. 2, 2021

(54) CRYOPRESERVATION

(71) Applicant: NantKwest, Inc., San Diego, CA (US)

(72) Inventors: Shannyn BESSETTE, San Diego, CA (US); Syed Raza ALI, San Diego, CA (US); Manju SAXENA, San Diego, CA (US)

(21) Appl. No.: 17/260,545

(22) PCT Filed: Jul. 9, 2019
(86) PCT No.: PCT/US2019/041028

§ 371 Date: Jan. 14, 2021

Related U.S. Application Data
(60) Provisional application No. 62/696,143, filed on Jul. 10, 2018.

(51) Int. Cl. A01N 1/02; C12N 5/0783

(52) U.S. Cl. CPC A01N 1/0221; C12N 5/0646

(57) ABSTRACT

Provided herein are methods and medium compositions for cryopreserving NK-92® cells.

### CROSS-REFERENCE TO RELATED APPLICATIONS

This application claims priority to U.S. Provisional Application No. 62/696,143, filed Jul. 10, 2018, which application is herein incorporated by reference for all purposes.

### SEQUENCE LISTING

The instant application contains a Sequence Listing which has been submitted electronically in ASCII format and is hereby incorporated by reference in its entirety. Said ASCII copy, created on Jul. 8, 2019, is named 104066-1146553-7610WO_SL.txt and is 3,757 bytes in size.

### BACKGROUND

Natural killer (NK) cells are cytotoxic lymphocytes that constitute a major component of the innate immune system. NK cells, generally representing about 10-15% of circulating lymphocytes, bind and kill targeted cells, including virus-infected cells and many malignant cells, non-specifically with regard to antigen and without prior immune sensitization. Herberman et al., Science 214:24 (1981). Killing of targeted cells occurs by inducing cell lysis. NK cells used for this purpose are isolated from the peripheral blood lymphocyte (“PBL”) fraction of blood from the subject, expanded in cell culture in order to obtain sufficient numbers of cells, and then re-infused into the subject. NK cells have been shown to be somewhat effective in both ex vivo therapy and in vivo treatment. However, such therapy is complicated by the fact that not all NK cells are cytolytic and the therapy is specific to the treated patient.

### BRIEF SUMMARY

Provided herein are methods for storing NK-92® cells in a cell freezing medium and compositions comprising NK-92® cells and the cell freezing medium.

Provided herein is a method of freezing NK-92® cells, the method comprising: contacting NK-92® cells with a cell freezing medium, wherein the cell freezing medium comprises a first composition and a second composition, wherein the first composition comprises: (a) one or more electrolytes selected from the group consisting of potassium ions, sodium ions, magnesium ions, and calcium ions; (b) one or more macromolecular agents selected from the group consisting of human albumin, polysaccharide and colloidal starch; (c) a pH buffer; (d) at least one sugar; (e) at least one sugar alcohol; (f) an impermeant agent selected from the group consisting of lactobionate, gluconate, citrate and glycerophosphate; and (g) DMSO; and wherein the second composition comprises albumin.

Optionally, the cells are contacted with the second composition before adding the first composition.

Optionally, the sugar alcohol is a 6-carbon sugar alcohol. Optionally, the sugar is a simple sugar. Optionally, the simple sugar is selected from the group consisting of glucose, dextran, dextrose, trehalose, and maltose.

Optionally, the albumin in the second composition is human albumin. Optionally, the second composition comprises 2-10% human albumin.

Optionally, the first and second composition are mixed at a volume ratio that ranges from 1:5 to 5:1, for example, about 1:1. Optionally, the first composition comprises 10% DMSO. Optionally, the first composition further comprises one or more substrates effective for regeneration of ATP. Optionally, the one or more substrates are selected from the group consisting of adenosine, fructose, ribose and adenine.

Optionally, the first composition further comprises glutathione. Optionally, the first composition further comprises one or more impermeant agents that can maintain ionic and osmotic balance during hypothermia.

Optionally, the cell freezing medium comprises less than 10% DMSO.

Optionally, the method further comprises freezing the cells using a controlled rate freeze to reach a final temperature of −80° C. or lower. Optionally, the method further comprises storing the cells at −80° C. to −196° C.

Optionally, the method further comprises thawing the preserved cells. Optionally, the cells are thawed at 37° C. Optionally, more than 70% of the cells are viable at the time of thawing. Optionally, more than 90% of the cells are viable at the time of thawing.

Optionally, the thawed cells have a direct cytoxicity of at least 80% against K562 cells at an effector to target ratio of 10:1. Optionally, the thawed cells have a direct cytoxicity that is 70-100% of that of the NK-92™ cells before the cells are frozen. Optionally, the thawed cells have a ADCC toxicity of at least 80-120% against Ramos cells at an effector to target ratio of 10:1, in the presence of an antibody targeting the Ramos cells. Optionally, the thawed cells have a ADCC cytoxicity that is 80-100% of that of the cells before the cells are frozen.

Optionally, the method further comprises administering the thawed cells to a patient in need via infusion.

Optionally, the NK-92® cells express a cytokine, Fc Receptor, chimeric antigen receptor, or a combination thereof.

Also provided is a NK-92® cell culture comprising: NK-92® cells and a cell freezing medium, wherein the cell freezing medium comprises a first composition and a second composition, wherein the first composition comprises: (a) one or more electrolytes selected from the group consisting of potassium ions, sodium ions, magnesium ions, and calcium ions; (b) one or more macromolecular agents selected from the group consisting of human albumin, polysaccharide and colloidal starch; (c) a pH buffer; (d) at least one sugar; (e) at least one sugar alcohol; (f) an impermeant agent being at least one member selected from the group consisting of lactobionate, gluconate, citrate and glycerophosphate; and (g) DMSO; and wherein the second composition comprises albumin. Optionally, the sugar alcohol is a 6-carbon sugar alcohol. Optionally, the simple sugar is selected from the group consisting of glucose, dextran, dextrose, trehalose, and maltose. Optionally, the second composition is human albumin. Optionally, the second composition is 2-10% human albumin.

Optionally, the first composition and second composition is mixed at a volume ratio ranging from 1:5 to 5:1, for example, about 1:1. Optionally, the first composition comprises 10% DMSO. Optionally, the first composition further comprises a substrate effective for the regeneration of ATP.

Optionally, the substrate in the first composition is selected from the group consisting of adenosine, fructose, ribose and adenine. Optionally, the first composition comprises 20-60 mM potassium ions. Optionally, the first composition comprises 50-150 mM sodium ions. Optionally, the first composition comprises 0.001-1 mM magnesium ions. Optionally, the first composition further comprises glutathione. Optionally, the cell culture is kept at 80° C. to −196° C. Optionally, at least 70% of NK-92® cells in the cell culture that has been kept at −80° C. to −196° C. are viable after cell culture is thawed.

Optionally, the NK-92® cells disclosed comprises a cytokine, Fc Receptor, chimeric antigen receptor, or a combination thereof.

Also provided is a cell freezing medium produced by mixing a first composition and a second composition at a one to one ratio, wherein the first composition comprises: (a) one or more electrolytes selected from the group consisting of potassium ions, sodium ions, magnesium ions, and calcium ions; (b) one or more macromolecular agents selected from the group consisting of human albumin, polysaccharide and colloidal starch; (c) a pH buffer; (d) at least one sugar; (e) at least one sugar alcohol; (f) an impermeant agent selected from the group consisting of lactobionate, gluconate, citrate and glycerophosphate; and (g) DMSO; and wherein the second composition comprises albumin.

The foregoing general description and the following detailed description are exemplary and explanatory and are intended to provide further explanation of the disclosure. Other objects, advantages and novel features will be readily apparent to those skilled in the art.

### DETAILED DESCRIPTION

After reading this description, it will become apparent to one skilled in the art how to implement various alternative embodiments and alternative applications. However, not all embodiments are described herein. It will be understood that the embodiments presented here are presented by way of an example only, and not limitation. As such, this detailed description of various alternative embodiments should not be construed to limit the scope or breadth of the disclosure as set forth herein. It is to be understood that the aspects described below are not limited to specific compositions, methods of preparing such compositions, or uses thereof as such may, of course, vary.

Unless defined otherwise, all technical and scientific terms used herein have the same meaning as commonly understood by one of ordinary skill in the art.

In this specification and in the claims that follow, reference will be made to a number of terms that shall be defined to have the following meanings:

The terminology used herein is for the purpose of describing particular embodiments only and is not intended to be limiting. As used herein, the singular forms “a,” “an” and “the” are intended to include the plural forms as well, unless the context clearly indicates otherwise. Thus, for example, reference to “a natural killer cell” includes a plurality of natural killer cells.

All numerical designations, e.g., pH, temperature, time, concentration, amounts, and molecular weight, including ranges, are approximations which are varied (-+) or (−) by increments of 0.1 or 1.0, where appropriate. It is to be understood, although not always explicitly stated, that all numerical designations may be preceded by the term “about.”

The term “about” when used in conjunction with a value means any value that is reasonably close to the value, i.e., within the range of ±10% of the value. In particular, it would include the value itself.

Unless otherwise noted, a percentage, when denoting a concentration, refer to a w/w percentage.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

It is also to be understood, although not always explicitly stated, that the reagents described herein are merely exemplary and that equivalents of such are known in the art.

“Optional” or “optionally” means that the subsequently described event or circumstance can or cannot occur, and that the description includes instances where the event or circumstance occurs and instances where it does not.

The term “comprising” is intended to mean that the compositions and methods include the recited elements, but not excluding others. “Consisting essentially of,” when used to define compositions and methods, shall mean excluding other elements of any essential significance to the combination. For example, a composition consisting essentially of the elements as defined herein would not exclude other elements that do not materially affect the basic and novel characteristic(s) of the claims. “Consisting of” shall mean excluding more than trace amount of other ingredients and substantial method steps. Embodiments defined by each of these transition terms are within the scope of the disclosure.

As used herein, “natural killer (NK) cells” are cells of the immune system that kill target cells in the absence of a specific antigenic stimulus, and without restriction according to major histocompatibility complex (MHC) class. Target cells may be cancer or tumor cells. NK cells are characterized by the presence of CD56 and the absence of CD3 surface markers.

As used herein, “NK-92® cells” refer to natural killer cells derived from the highly potent unique cell line described in Gong et al. (1994), rights to which are owned by NantKwest.

As used herein, the term “aNK™ cells” refers to unmodified natural killer cells derived from the highly potent unique cell line described in Gong et al. (1994), rights to which are owned by NantKwest.

As used herein, the term “haNK® cells” refers to natural killer cells derived from the highly potent unique cell line described in Gong et al. (1994), rights to which are owned by NantKwest, modified to express CD16 on the cell surface (hereafter, “CD16+ NK-92™ cells” or “haNK® cells”).

As used herein, the term “taNK® cells” refers to natural killer cells derived from the highly potent unique cell line described in Gong et al. (1994), rights to which are owned by NantKwest, modified to express a chimeric antigen receptor (hereafter, “CAR-modified NK-92™ cells” or “taNK® cells”).

As used herein, the term “t-haNK™ cells” refers to NK-92® cells that have been engineered to express an Fc receptor and a chimeric antigen receptor (CAR) with affinity for a cancer specific antigen, a cancer associated antigen, or a tumor specific antigen. For example, the tumor specific antigen is CD19, and these NK-92® cells are referred to as CD19 t-haNK™ cells.

The term “Fc receptor” refers to a protein found on the surface of certain cells (e.g., natural killer cells) that contribute to the protective functions of the immune cells by binding to part of an antibody known as the Fc region. Binding of the Fc region of an antibody to the Fc receptor (FcR) of a cell stimulates phagocytic or cytotoxic activity of a cell via antibody-mediated phagocytosis or antibody-dependent cell-mediated cytotoxicity (ADCC). FcRs are classified based on the type of antibody they recognize. For example, Fc-gamma receptors (FcγR) bind to the IgG class of antibodies. FcγRIII-A (also called CD16) is a low affinity Fc receptor bind to IgG antibodies and activate ADCC. FcγRIII-A are typically found on NK cells. NK-92® cells do not express FcγRIII-A.

The term “chimeric antigen receptor” (CAR), as used herein, refers to an extracellular antigen-binding domain that is fused to an intracellular signaling domain. CARs can be expressed in T cells or NK cells to increase cytotoxicity. In general, the extracellular antigen-binding domain is a scFv that is specific for an antigen found on a cell of interest. A CAR-expressing NK-92® cell is targeted to cells expressing certain antigens on the cell surface, based on the specificity of the scFv domain. The scFv domain can be engineered to recognize any antigen, including tumor-specific antigens.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, a polynucleotide encompasses both the double-stranded form and each of two complementary single-stranded forms known or predicted to make up the double-stranded form.

The term “expression” refers to the production of a gene product. The term “transient” when referred to expression means a polynucleotide is not incorporated into the genome of the cell.

The term “cytokine” or “cytokines” refers to the general class of biological molecules which effect cells of the immune system. Exemplary cytokines include, but are not limited to, interferons and interleukins (IL), in particular IL-2, IL-12, IL-15, IL-18 and IL-21. In preferred embodiments, the cytokine is IL-2.

As used herein, the term “vector” refers to a non-chromosomal nucleic acid comprising an intact replicon such that the vector may be replicated when placed within a permissive cell, for example by a process of transformation. A vector may replicate in one cell type, such as bacteria, but have limited ability to replicate in another cell, such as mammalian cells. Vectors may be viral or non-viral. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

As used herein, the term “substantially the same”, used interchangeably with the term “comparable”, or “similar”, when referring to cytotoxicity, viability or cell recovery, refers to the that the two measurements of cytotoxicity, viability or cell doubling time are no more than 25%, no more than 20%, no more than 15% different, no more than 10%, no more than 8%, or no more than 5% different from each other.

As used herein, the terms “cytotoxic” when used to describe the activity of effector cells such as NK cells, relates to killing of target cells by any of a variety of biological, biochemical, or biophysical mechanisms.

Titles or subtitles may be used in the specification for the convenience of a reader, which are not intended to influence the scope of the present disclosure. Additionally, some terms used in this specification are more specifically defined below.

NK-92® cells have previously been evaluated as a therapeutic agent in the treatment of certain cancers. Unlike NK cells, NK-92® is a cytolytic cancer cell line which was discovered in the blood of a subject suffering from a non-Hodgkins lymphoma and then immortalized ex vivo. NK-92® cells lack the major inhibitory receptors that are displayed by normal NK cells, but retain the majority of the activating receptors. NK-92® cells do not, however, attack normal cells nor do they elicit an unacceptable immune rejection response in humans. Characterization of the NK-92® cell line is disclosed, e.g., in WO 1998/49268 and U.S. Pat. No. 8,034,332.

However, the storage of the NK-92® cells remains a challenge. For storage, NK-92® cells are typically stored in freezing media and thawed before use. The freeze-thaw procedures induce molecular cell stress responses, causing apoptosis and/or necrosis upon; and the formation of intracellular ice can have significant negative effect on cell viability. As a result, the cell viability is often low, which impairs the therapeutic effect of the NK-92® cells. Further, the existing, routine freezing medium uses a high concentration of DMSO, typically 10%, which can also decrease viability of the cells. In addition, many of these traditional cell freezing medium comprises components that are not suitable for direct infusion to patients. After thawing, the cells must be cultured and reformulated in a medium that is suitable for infusion. This requisite reformulation step would increase the cost for the therapy and also, could introduce human operational error. Thus, the needs remain for an economical, cell freezing medium that can be used to store NK-92® cells with minimized damages to cells, and the NK-92® cells after thawing can be directly administered to patients.

This disclosure provides a cell freezing medium for cryopreserving NK-92® cells, which comprises a first composition and a second composition. As compared to various existing freezing media, the cell freezing medium of this disclosure can mitigate temperature-induced molecular cell stress responses during freezing and thawing. It can be used to cryopreserve a broad spectrum of cell types, including NK-92™ cells. The medium is much more effective in reducing post-preservation necrosis and apoptosis as compared to commercial and home-brew isotonic and extracellular formulations. This enables greatly improved post-thaw cell yield, viability, and recovery. Optionally, one or more or all ingredients in the first and/or second compositions meet the USP standard. Optionally, these ingredients are manufactured under cGMP and suitable for therapeutic uses.

The first composition disclosed herein comprises one or more electrolytes. Exemplary electrolytes that can be used in the first composition may be one or more of potassium ions, sodium ions, magnesium ions, and calcium ions. Optionally, potassium ions are present in the first composition in a concentration ranging from 35-45 mM, sodium ions are present in a concentration ranging from 80-120 mM, magnesium ions are present in a concentration ranging from 2-10 mM, and calcium ions present in a concentration ranging from 0.01-0.1 mM.

The first composition disclosed herein also comprises one or more marcomolecualr agents. Exemplary macromolecular agents that can be used in the first composition may be one or more of polysaccharide and colloidal starch. Optionally, the first composition comprises human albumin.

The first composition may comprise a pH buffer that is effective under physiological and hypothermic conditions. Non-limiting examples of suitable pH buffers include dihydrogen phosphate (H₂PO₄⁻), bicarbonate ion (HCO₃⁻) and HEPES. Optionally, H₂PO₄⁻ is present in an amount of 5-15 mmol/L, e.g., 10.0 mmol/L, HCO₃⁻ is present in an amount of 2.5-10 mmol/L, e.g., 5.0 mmol/L, and/or HEPES is present in an amount of 15-35 mmol/L.

The first composition may also comprise one or more sugars. Optionally, the one or more sugars comprises sucrose. Optionally, the one or more sugar is a simple sugar that is selected from the group consisting of glucose, dextran, dextrose, trehalose, and maltose. Optionally, the glucose is present in the first composition at a concentration of 2.0-10.0 mmol/L, e.g., 3.0-7.0 mmol/L, or 5.0 mmol/L.

The first composition may also comprise one or more sugar alcohols. Each of the sugar alcohols can be a 3-carbon, 4-carbon, 5-carbon, 6-carbon, 7-carbon, 12-carbon, 18-carbon, or 24-carbon sugar alcohol. Optionally, the sugar alcohol is a 6-carbon sugar alcohol. Non-limiting examples of 6-carbon sugar alcohols include mannitol, sorbitol, galactitol, fucitol, iditol, and inositol. Optionally, the 6-carbon sugar alcohol is mannitol. Non-limiting examples of 3-carbon sugar alcohols include glycerol, erythritol, and threitol. Non-limiting examples of 5-carbon sugar alcohols include arabitol, xylitol, ribitol. Non-limiting examples of 7-carbon sugar alcohols include volemitol. Non-limiting examples of 12-carbon sugar alcohols include isomalt, maltitol, and lactitol. Non-limiting examples of 18-carbon sugar alcohols include maltotriitol. Non-limiting examples of 24-carbon sugar alcohols include maltotetraitol. Optionally, the one or more sugar alcohols are present in the the first composition in a total amount of 10-30 mmol/L, e.g., 20.0 mmol/L.

The first composition may also comprise one or more impermeant agents to maintain ionic and osmotic balance within body tissues during hypothermia. These impermeant agents is included to balance the fixed ions inside cells that are responsible for ocotic pressure leading to osmotic cell swelling and eventual lysis during hypothermia. Non-limiting examples of impermeant agents include lactobionate, gluconate, citrate and glycerophosphate. Lactobionate, for example, is a strong chelator of calcium and iron and may contribute to minimizing cell injury due to calcium influx and free radical formation. Optionally, the one or more impermeant agents are present in a total concentration of 40-200 mmol/L, e.g., 50-150 mmol/L, e.g., 100.0 mmol/L.

The first composition also comprises DMSO. Optionally, the DMSO is USP grade DMSO. The USP is a chemical grade of sufficient purity to meet or exceed requirements of the United States Pharmacopeia (USP) and is acceptable for food, drug, or medical use. Optionally, the first composition comprises 2-18% v/v DMSO, e.g., 4-14%, 6-12%, 6-16%, or about 10% v/v DMSO.

Optionally, the first composition comprises a substrate effective for regeneration of ATP. For example, the substrate may be one that is selected from the group consisting of adenosine, fructose, ribose and adenine. Optionally, the first composition comprises adenosine. Optionally adenosine is present in a concentration of 0.5- 5 mmol/L, e.g., 1-4 mmol/L, 1-3 mmol/L, or about 2.0 mmol/L.

Optionally, the first composition comprises a cellular antiboxidant, such as glutathione. In addition to acting as an important cellular antioxidant, glutathione is also a cofactor for glutathione peroxidase, which enables metabolism of lipid perioxides and hydrogen peroxide. Optionally, the first composition comprises 0.5-5 mmol/L of the cellular antioxidant, e.g., 1-4 mmol/L, 2-5 mmol/L, or about 3.0 mmol/L. For the purpose of this disclosure, the substrate and the antioxidant used in the medium are collectively referred to as the metabolites.

Suitable medium for use as the first composition include Cryostor® CS10, available from Biolife Solutions, Bothell, Wash.

In one illustrative embodiment, the first composition comprises the following ingredients as shown in Table 1 below

TABLE 1

| | Components | Concentrations |
|---|---|---|
| Electrolytes | Na⁺ | 100.0 mmol/L |
| | K⁺ | 42.5 mmol/L |
| | Ca²⁺ | 0.05 mmol/L |
| | Mg²⁺ | 5.0 mmol/L |
| | Cl⁻ | 17.1 mmol/L |
| pH buffers | H₂PO₄⁻ | 10.0 mmol/L |
| | HCO₃⁻ | 5.0 mmol/L |
| | HEPES | 25.0 mmol/L |
| Impermeants | Lactobionate | 100.0 mmol/L |
| Sugar alcohol | Mannitol | 20.0 mmol/L |
| Sugars | Glucose | 5.0 mmol/L |
| | Dextran-40 | 6.0% |
| | Sucrose | 20.0 mmol/L |
| Metabolites | Adenosine | 2.0 mmol/L |
| | Glutathione | 3.0 mmol/L |
| DMSO | DMSO | 10% |

In this particular embodiment, the first composition has an osmolarity of 350 mOsm/kg and a pH of 7.6 (at 25° C.).

The second composition used in the cell freezing medium include albumin. Albumin is a protein supplement in cell culture used to deliver unesterified fatty acids into and from cells; albumin can be, for example, human albumin, such as human serum albumin (HSA) and human plasma albumin. Albumin is beneficial to the overall health of the cryopreserved cells. Optionally, the albumin is human albumin. Optionally, the albumin is human serum albumin or plasma derived human albumin. Human albumin (“HA”) is the most copious protein in human serum at approximately 3.5-5.0 g/dL and functions as a carrier protein for steroids and fatty acids in blood. Human albumin is commercially available, for example, from CSL Behring. Optionally, the second composition comprises 1-10% w/v HA, e.g., 2-10%, 2-8%. 3-6%, or 5.0% w/v. As discussed below, including HA in the cell freezing medium not only is beneficial for maintaining cell morphology and function, it can also ease manufacturing process.

The cell freezing medium disclosed herein is made by mixing the first composition and the second composition. During the mixing, the DMSO in the first composition is diluted. Thus, the cell freezing medium typically comprises a low DMSO concentration, e.g., less than 10%. In general, DMSO, although widely used in cell freezing medium, is cytotoxic; it can alter cell morphology, reduce membrane integrity, and reduce cell viability, the toxic effect worsens as the concentration of DMSS increases in the medium. Thus, the claimed methods can maintain the DMSO at a relatively low concentration, e.g., at a concentration that is lower than 10%, which can improve post-thaw cell viability and function. Optionally, the first composition and the second composition in the cell freezing medium are mixed in a volume ratio of 1:5 to 5:1, e.g., 1:3 to 3:1, 1:2 to 2:1, or about 1:1. Optionally, the cell freezing medium comprises about 5, about 6, about 7, about 8, about 9, or about 10% v/v DMSO.

Thus, as further disclosed below, the NK-92® cells that have been freezed in cryopreseravion medium comprising the first composition and the second composition disclosed herein shows improved viability, better cytotoxicity, and/or ADCC than cells preserved in other media and are suitable for direct infusion to a patient in need.

This disclosure provides methods of freezing NK-92® cells using the novel cell freezing medium disclosed herein. In some cases, NK-92® cells can be harvested from culture by centrifugation. In some cases, the cell culture before centrifugation contains 2-10%, e.g., about 5% HA. In some cases, the cell culture before harvesting contains about the same concentration of the HA as in the second composition. Optionally the centrifugation is performed in a kSep®400 centrifuge that are aseptically attached to a culture vessel, e.g., Xuri bags. In some cases, harvested cells are resuspended in the second composition before adding the first composition. As the cell culture before harvesting typically already contains HA, using the second composition, which also comprises HA, keeps cells in a similar medium environment and thus minimizes negative impact of harvesting on the morphology and function of the cells. The methods dispense the need for washing cells after harvesting, an extra step often required when before freezing cells in a conventional cell freezing medium (which typically has different medium composition from the pre-harvesting medium). Thus, the claimed method significantly reduces processing time, reduces operational errors and increases cell recovery.

Optionally, the cells in the cell freezing medium are in a concentration that is suitable for clinical applications. For example, the NK-92® cells may be in a concentration of 1.8-2.4×10e7 cells/mL, e.g., 1.8×10e7 cells/mL, or 2×10e7 cells/mL. Optionally, the NK-92™ cells are in the cell freezing medium in a volume that is suitable for infusion to patients. For example, the volume may be 55-100 mL

NK-92™ cells in the cell freezing medium may be frozen using methods well known in the art. In one illustrative example, the NK-92® cells are frozen using a controlled-rate freezing program, until the temperature reaches −70° C. to −196° C., e.g., −80° C. to −196° C. Frozen cells are then stored in vapor phase of a liquid N₂ cryofreezer.

Cells can be thawed by any methods that are suitable for thawing cells. Optionally, the cells can be thawed at 37° C., in a device such as a waterbath or ViaThaw control rate thawing unit from Asymptote, Cambridge, United Kingdom.

Optionally, the viability of the thawed cells is assessed. Methods for measuring cell viability are also well known, for example, trypan blue exclusion assay, in which dead cells are stained blue and viable cell number can be calculated by subtracting the trypan blue stained cells from the total cells. Cell counting can be performed on a counting chamber of a hemacytometer. Automatic cell counting, based on the fact that cells show great electrical resistance, are also commonly used to count cells as well as measure their volume. One example of the automatic cell counter is the Coulter counter, available from Beckman Coulter. Another example of the automatic cell counter is NucleoCounter® NC-200™, available from Chemometec. The NK-92™ cells that have been freezed in the cell freezing medium described herein and thawed may have 80-100%, e.g., 85-100%, 90-100%, 92-100% viability. Optionally, the NK-92™ cells that have been freezed and thawed may have viability that is at least 90%, at least 91%, at least 92%, at least 93%, at least 94% of that of the cells before freezing.

Optionally, the cytotoxicity of the thawed NK-92® cells can be assessed. Direct cytotoxicity of the produced NK-92® cells, the ability to target and kill aberrant cells, such as virally infected and tumorigenic cells, can be assessed by methods well known in the art, for example, a ⁵¹Cr release assay (Gong et al. (1994)) using the procedure described by Klingemann et al. (Cancer Immunol. Immunother. 33:395-397 (1991)). The percentage of specific cytotoxicity can be calculated based on the amount of released ⁵¹Cr. See Patent Pub. No. US20020068044.

Alternatively, direct cytotoxicity of the produced NK-92® cells can also be assessed using a calcein release assay. For example, the NK 92® cells (referred to as the effector in the assay) can be mixed with the calcein loaded target cells (referred to as target in the assay) at certain ratios. After incubation for a period of time (e.g., 2-8 hours, or about 3 hours) the calcein released from the target cells can be assessed, e.g., by a fluorescence plate reader. The ratio of the effector and target used in the assay may vary, optionally the effector: target ratio may be 20:1, 15:1, 10:1, 8:1, or 5:1; preferably the effector: target ratio is 10:1. The target cells can be any cells that express MHC molecules that can be recognized by the NK-92® cells, for example, K562 cells, or BT-474 cells. The values of cytotoxicity of NK-92™ cells may vary depending on the type of target cells used as well as the effector:target ratio and the growth cycle which the NK-92® cells are in.

The NK-92™ cells after thawing has surprisingly retained the substantial portion of the cytotoxicity; in some cases, the thawed NK-92® cells at least 70-100%, e.g., 80-100%, at 90-100% of the cytotoxicity of that of the NK-92® cells before the cells are frozen (pre-freeze state). Optionally, the thawed NK-92® cells have 70-100%, e.g., 75-95%, 70-90%, or about 88%, direct cytotoxicity, when using a calcein release assay as described below. Optionally, the effect:target ratio is 10:1 and the target cells being K562 cells.

Optionally, the cytotoxicity of NK-92® cells, e.g., haNK® cells, that is assessed is the antibody dependent cytotoxicity (ADCC). Methods for measuring the ADCC of NK-92® cells are similar to the methods of measuring direct cytotoxicity as described above except that an antibody that can recognize the target cell is added. The Fc receptor of the NK cells recognizes the cell-bound antibodies and triggers cytolytic reaction and killing the target cells. In one illustrative example, the haNK® cells can be incubated with Rituxan (an antibody) and Ramos (target cells) and killing of the Ramos cells can be measured by the release of internal components of the target cells, e.g., ⁵¹Cr or calcein, as described above. The ratio of the effector and target used in the assay may vary, optionally the effector:target ratio may be 20:1, 15:1, 10:1, 8:1, or 5:1; preferably the effector:target ratio is 10:1. in some cases, the thawed haNK® cells have 70-100%, e.g., 80-100%, at 90-100% of the cytotoxicity of that of the NK-92™ cells before the cells are frozen (pre-freeze state). HaNK® cells after thawing may have a ADCC cytotoxicity of 80-120%, e.g., 90-115%, 97-110%, or 100-120% when using an effector:target ratio of 10:1. In some embodiments, the haNK® cells after thawing may have a ADCC cytotoxicity that is 80-100% of that of the haNK® cells before the cells are frozen.

Optionally, the phenotypes of the thawed cells are assessed by assaying the expression level of one or more surface markers, optionally from of CD54, CD56, NKG2D, NKp30, CD3, and CD16. The marker expression can be determined using methods well known in the art, e.g., by staining cells with specific fluorochrome-conjugated antibodies and detecting bound antibodies by flow cytometry. The freeze and thaw of NK-92® cells in the cell freezing medium disclosed herein does not alter the expression of key surface makers. For example, the NK-92® cells freezed and thawed as described above retained expression of surface markers typical of an NK cell in an early differentiation stage, such as a number of activation receptors including NKG2D and NKp30, but lacking FcγRIIIa (CD16). The thawed NK-92™ cells also express CD56 and CD54. Optionally, greater than 90% of the cells in the population of the thawed cells express CD56, CD54, NKG2D, or NKp30 and less than 5% of the cells in the population of cells express CD3 or CD16.

The NK-92® cells that can be cultured using the methods disclosed herein include aNK™ cells, haNK® cells, and taNK® cells, which are further described below.

For purposes of this invention and unless indicated otherwise, the term “NK-92®” or “NK92®” is intended to refer to the original NK-92® cell lines as well as NK-92® cell lines, clones of NK-92® cells, and NK-92® cells that have been modified (e.g., by introduction of exogenous genes). NK-92® cells and exemplary and non-limiting modifications thereof are described in U.S. Pat. Nos. 7,618,817; 8,034,332; 8,313,943; 9,181,322; 9,150,636; and published U.S. application Ser. No. 10/008,955, all of which are incorporated herein by reference in their entireties, and include wild type NK-92®, NK-92®-CD16, NK-92®-CD16-γ, NK-92®-CD16-ζ, NK-92®-CD16(F176V), NK-92®MI, and NK-92®CI. NK-92® cells are known to persons of ordinary skill in the art, to whom such cells are readily available from NantKwest, Inc.

The NK-92® cell line is a unique cell line that was discovered to proliferate in the presence of interleukin 2 (IL-2). Gong et al., Leukemia 8:652-658 (1994). These cells have high cytolytic activity against a variety of cancers. The NK-92® cell line is a homogeneous cancerous NK cell population having broad anti-tumor cytotoxicity with predictable yield after expansion. Phase I clinical trials have confirmed its safety profile. NK-92® was discovered in the blood of a subject suffering from a non-Hodgkins lymphoma and then immortalized ex vivo. NK-92® cells are derived from NK cells, but lack the major inhibitory receptors that are displayed by normal NK cells, while retaining the majority of the activating receptors. NK-92® cells do not, however, attack normal cells nor do they elicit an unacceptable immune rejection response in humans. Characterization of the NK-92® cell line is disclosed in WO 1998/49268 and U.S. Patent Application Publication No. 2002-0068044.

The NK-92® cell line is found to exhibit the CD56^{bright}, CD2, CD7, CD11a, CD28, CD45, and CD54 surface markers. It furthermore does not display the CD1, CD3, CD4, CD5, CD8, CD10, CD14, CD16, CD19, CD20, CD23, and CD34 markers. Growth of NK-92® cells in culture is dependent upon the presence of recombinant interleukin 2 (rIL-2), with a dose as low as 1 IU/mL being sufficient to maintain proliferation. IL-7 and IL-12 do not support long-term growth, nor do other cytokines tested, including IL-1α, IL-6, tumor necrosis factor α, interferon α, and interferon γ. NK-92® has high cytotoxicity even at a low effector:target (E:T) ratio of 1:1. Gong, et al., supra. NK-92™ cells are deposited with the American Type Culture Collection (ATCC), designation CRL-2407.

Heretofore, studies on endogenous NK cells have indicated that IL-2 (1000 IU/mL) is critical for NK cell activation during shipment, but that the cells need not be maintained at 37° C. and 5% carbon dioxide. Koepsell, et al., Transfusion 53:398-403 (2013).

Modified NK-92® cells are known and include, but are not limited to, those described in, e.g., U.S. Pat. Nos. 7,618,817, 8,034,332, and 8,313,943, U.S. Patent Application Publication No. 2013/0040386, all of which are incorporated herein by reference in their entireties, such as wild type NK-92®, NK-92®-CD16, NK-92®-CD16-γ, NK-92®-CD16-ζ, NK-92®-CD16(F157V), NK-92®mi and NK-92®ci.

Although NK-92® cells retain almost all of the activating receptors and cytolytic pathways associated with NK cells, they do not express CD16 on their cell surfaces. CD16 is an Fc receptor which recognizes and binds to the Fc portion of an antibody to activate NK cells for antibody-dependent cellular cytotoxicity (ADCC). Due to the absence of CD16 receptors, NK-92™ cells are unable to lyse target cells via the ADCC mechanism and, as such, cannot potentiate the anti-tumor effects of endogenous or exogenous antibodies (i.e., rituxan and herceptin).

Studies on endogenous NK cells have indicated that IL-2 (1000 IU/mL) is critical for NK cell activation during shipment, but that the cells need not be maintained at 37° C. and 5% carbon dioxide. Koepsell, et al., Transfusion 53:398-403 (2013). However, endogenous NK cells are significantly different from NK-92′⁻'¹) cells, in large part because of their distinct origins: NK-92® is a cancer-derived cell line, whereas endogenous NK cells are harvested from a donor (or the patient) and processed for infusion into a patient. Endogenous NK cell preparations are heterogeneous cell populations, whereas NK-92® cells are a homogeneous, clonal cell line. NK-92® cells readily proliferate in culture while maintaining cytotoxicity, whereas endogenous NK cells do not. In addition, an endogenous heterogenous population of NK cells does not aggregate at high density. Furthermore, endogenous NK cells express Fc receptors, including CD-16 receptors that are not expressed by NK-92® cells.

### Fc Receptors

Fc receptors bind to the Fc portion of antibodies. Several Fc receptors are known, and differ according to their preferred ligand, affinity, expression, and effect following binding to the antibody.

TABLE 2
Illustrative Fc receptors

| | Principal | Affinity | | |
|---|---|---|---|---|
| | antibody | for | | Effect following binding to |
| Receptor name | ligand | ligand | Cell distribution | antibody |
| FcγRI (CD64) | IgG1 and | High | Macrophages | Phagocytosis |
| | IgG3 | (Kd ~ | Neutrophils | Cell activation |
| | | 10⁻⁹ M) | Eosinophils | Activation of respiratory |
| | | | Dendritic cells | burst |
| | | | | Induction of microbe |
| | | | | killing |
| FcγRIIA (CD32) | IgG | Low | Macrophages | Phagocytosis |
| | | (Kd > | Neutrophils | Degranulation (eosinophils) |
| | | 10⁻⁷ M | Eosinophils | |
| | | | Platelets | |
| | | | Langerhans cells | |
| FcγRIIB1 (CD32) | IgG | Low | B Cells | No phagocytosis |
| | | (Kd > | Mast cells | Inhibition of cell activity |
| | | 10⁻⁷ M) | | |
| FcγRIIB2 (CD32) | IgG | Low | Macrophages | Phagocytosis |
| | | (Kd > | Neutrophils | Inhibition of cell activity |
| | | 10⁻⁷ M) | Eosinophils | |
| FcγRIIIA (CD16a) | IgG | Low | NK cells | Induction of antibody- |
| | | (Kd > | Macrophages (certain | dependent cell-mediated |
| | | 10⁻⁶ M) | tissues) | cytotoxicity (ADCC) |
| | | | | Induction of cytokine |
| | | | | release by macrophages |
| FcγRIIIB (CD16b) | IgG | Low | Eosinophils | Induction of microbe |
| | | (Kd > | Macrophages | killing |
| | | 10⁻⁶ M) | Neutrophils | |
| | | | Mast cells | |
| | | | Follicular dendritic | |
| | | | cells | |
| FcεRI | IgE | High | Mast cells | Degranulation |
| | | (Kd ~ | Eosinophils | Phagocytosis |
| | | 10⁻¹⁰ M) | Basophils | |
| | | | Langerhans cells | |
| | | | Monocytes | |
| FcεRII (CD23) | IgE | Low | B cells | Possible adhesion molecule |
| | | (Kd > | Eosinophils | IgE transport across human |
| | | 10⁻⁷ M) | Langerhans cells | intestinal epithelium |
| | | | | Positive-feedback |
| | | | | mechanism to enhance |
| | | | | allergic sensitization (B |
| | | | | cells) |
| FcαRI (CD89) | IgA | Low | Monocytes | Phagocytosis |
| | | (Kd > | Macrophages | Induction of microbe |
| | | 10⁻⁶ M) | Neutrophils | killing |
| | | | Eosinophils | |
| Fcα/μR | IgA and IgM | High for | B cells | Endocytosis |
| | | IgM, | Mesangial cells | Induction of microbe |
| | | Mid for | Macrophages | killing |
| | | IgA | | |
| FcRn | IgG | | Monocytes | Transfers IgG from a |
| | | | Macrophages | mother to fetus through the |
| | | | Dendritic cells | placenta |
| | | | Epithelial cells | Transfers IgG trom a |
| | | | Endothelial cells | mother to infant in milk |
| | | | Hepatocytes | Protects IgG from |
| | | | | degradation |

In some embodiments NK-92™ cells are modified to express an Fc receptor protein on the cell surface.

In some embodiments, the Fc receptor is CD16. A representative amino acid sequence encoding CD16 is shown in SEQ ID NO:2. A representative polynucleotide sequence encoding CD16 is shown in SEQ ID NO:1. In some embodiments, NK-92™ cells are modified by introducing a polynucleotide encoding a CD16 polypeptide has at least about 70% polynucleotide sequence identity with a polynucleotide sequence encoding a full-length, including signal peptide, naturally occurring CD16 that has a phenylalanine at position 176 of the full-length CD16. In some embodiments, a polynucleotide encoding a CD16 polypeptide has at least about 70% polynucleotide sequence identity with a polynucleotide sequence encoding a full-length, including the signal peptide, naturally occurring CD16 that has a valine at position 176.

Homologous polynucleotide sequences include those that encode polypeptide sequences coding for variants of CD16. In some embodiments, homologous CD16 polynucleotides may be about 150 to about 700, about 750, or about 800 polynucleotides in length, although CD16 variants having more than 700 to 800 polynucleotides are within the scope of the disclosure.

In other examples, cDNA sequences having polymorphisms that change the CD16 amino acid sequences are used to modify the NK-92® cells, such as, for example, the allelic variations among individuals that exhibit genetic polymorphisms in CD16 genes. In other examples, CD16 genes from other species that have a polynucleotide sequence that differs from the sequence of human CD16 are used to modify NK92™ cells.

In examples, variant polypeptides are made using methods known in the art such as oligonucleotide-mediated (site-directed) mutagenesis, alanine scanning, and PCR mutagenesis. Site direct mutagenesis (Carter, 1986; Zoller and Smith, 1987), cassette mutagenesis, restriction selection mutagenesis (Wells et al., 1985) or other known techniques can be performed on the cloned DNA to produce CD16 variants (Ausubel, 2002; Sambrook and Russell, 2001).

Conservative substitutions in the amino acid sequence of human CD16 polypeptide, whereby an amino acid of one class is replaced with another amino acid of the same class, fall within the scope of the disclosed CD16 variants as long as the substitution does not materially alter the activity of the polypeptide. Conservative substitutions are well known to one of skill in the art. Non-conservative substitutions that affect (1) the structure of the polypeptide backbone, such as a β-sheet or α-helical conformation, (2) the charge, (3) the hydrophobicity, or (4) the bulk of the side chain of the target site can modify CD16 polypeptide function or immunological identity. Non-conservative substitutions entail exchanging a member of one of these classes for another class. Substitutions may be introduced into conservative substitution sites or more preferably into non-conserved sites.

In some embodiments, CD16 polypeptide variants are at least 200 amino acids in length and have at least 70% amino acid sequence identity, or at least 80%, or at least 90% identity to SEQ ID NO:1 or SEQ ID NO:2. In some embodiments, CD16 polypeptide variants are at least 225 amino acid in length and have at least 70% amino acid sequence identity, or at least 80%, or at least 90% identity to SEQ ID NO:1 or SEQ ID NO:2.

In some embodiments a nucleic acid encoding a CD16 polypeptide may encode a CD16 fusion protein. A CD16 fusion polypeptide includes any portion of CD16 or an entire CD16 fused with a non-CD16 polypeptide. In some embodiment, a fusion polypeptide may be created in which a heterologous polypeptide sequence is fused to the C-terminus of CD16 or is positioned internally in the CD16. Typically, up to about 30% of the CD16 cytoplasmic domain may be replaced. Such modification can enhance expression or enhance cytotoxicity (e.g., ADCC responsiveness). In other examples, chimeric proteins, such as domains from other lymphocyte activating receptors, including but not limited to Ig-a, Ig-B, CD3-e, CD3-d, DAP-12 and DAP-10, replace a portion of the CD16 cytoplasmic domain.

Fusion genes can be synthesized by conventional techniques, including automated DNA synthesizers and PCR amplification using anchor primers that give rise to complementary overhangs between two consecutive gene fragments that can subsequently be annealed and re-amplified to generate a chimeric gene sequence (Ausubel, 2002). Many vectors are commercially available that facilitate sub-cloning CD16 in-frame to a fusion moiety.

### Chimeric Antigen Receptor

As described herein, NK-92™ cells are further engineered to express a chimeric antigen receptor (CAR) on the cell surface. Optionally, the CAR is specific for a tumor-specific antigen. Tumor-specific antigens are described, by way of non-limiting example, in U.S. 2013/0189268; WO 1999024566 A1; U.S. Pat. No. 7,098,008; and WO 2000020460 A1, each of which is incorporated herein by reference in its entirety. Tumor-specific antigens include, without limitation, NKG2D, CS1, GD2, CD138, EpCAM, EBNA3C, GPA7, CD244, CA-125, ETA, MAGE, CAGE, BADE, HAGE, LAGE, PAGE, NY-SEO-1, GAGE, CEA, CD52, CD30, MUC5AC, c-Met, EGFR, FAB, WT-1, PSMA, NY-ESO1, AFP, CEA, CTAG1B, CD19 and CD33. Additional non-limiting tumor-associated antigens, and the malignancies associated therewith, can be found in Table 3.

TABLE 3
Tumor-Specific Antigens and Associated Malignancies

| Target Antigen | Associated Malignancy |
|---|---|
| α-Folate Receptor | Ovarian Cancer |
| CAIX | Renal Cell Carcinoma |
| CD19 | B-cell Malignancies |
| | Chronic lymphocytic leukemia (CLL) |
| | B-cell CLL (B-CLL) |
| | Acute lymphoblastic leukemia (ALL); ALL |
| | post Hematopoietic stem cell transplantation |
| | (HSCT) |
| | Lymphoma; Refractory Follicula |
| | Lymphoma; B-cell non-Hodgkin lymphoma |
| | (B-NHL) |
| | Leukemia |
| | B-cell Malignancies post-HSCT |
| | B-lineage Lymphoid Malignancies post |
| | umbilical cord blood transplantation (UCBT) |
| CD19/CD20 | Lymphoblastic Leukemia |
| CD20 | Lymphomas |
| | B-Cell Malignancies |
| | B-cell Lymphomas |
| | Mantle Cell Lymphoma |
| | Indolent B-NHL |
| | Leukemia |
| CD22 | B-cell Malignancies |
| CD30 | Lymphomas; Hodgkin Lymphoma |
| CD33 | AML |
| CD44v7/8 | Cervical Carcinoma |
| CD138 | Multiple Myeloma |
| CD244 | Neuroblastoma |
| CEA | Breast Cancer |
| | Colorectal Cancer |
| CS1 | Multiple Myeloina |
| EBNA3C | EBV Positive T-cells |
| EGP-2 | Multiple Malignancies |
| EGP-40 | Colorectal Cancer |
| EpCAM | Breast Carcinoma |
| Erb-B2 | Colorectal Cancer |
| | Breast Cancer and Others |
| | Prostate Cancer |
| Erb-B 2,3,4 | Breast Cancer and Others |
| FBP | Ovarian Cancer |
| Fetal Acetylcholine | Rhabdomyosarcoma |
| Receptor | |
| GD2 | Neuroblastoma |
| GD3 | Melanoma |
| GPA7 | Melanoma |
| Her2 | Breast Carcinoma |
| | Ovarian Cancer |
| | Tumors of Epithelial Origin |
| Her2/new | Medulloblastoma |
| | Lung Malignancy |
| | Advanced Osteosarcoma |
| | Glioblastoma |
| IL-13R-a2 | Glioma |
| | Glioblastoma. |
| | Medulloblastoma |
| KDR | Tumor Neovasculature |
| k-light chain | B-cell Malignancies |
| | B-NHL , CLL |
| LeY | Carcinomas |
| | Epithelial Derived Tumors |
| L1 Cell Adhesion | Neuroblastoma |
| Molecule | |
| MAGE-A1 | Melanoma |
| Mesothelin | Various Tumors |
| MUC1 | Breast Cancer; Ovarian Cancer |
| NKG2D Ligands | Various Tumors |
| Oncofetal Antigen (h5T4) | Various Tumors |
| PSCA | Prostate Carcinoma. |
| PSNLA | Prostate/Tumor Vasculature |
| TAA Targeted by mAb | Various Tumors |
| IgE | |
| TAG-72 | Adenocarcinomas |
| VEGF-R2 | Tumor -Neovasculature |

In some embodiments, the CAR targets CD19, CD33 or CSPG-4.

In examples, variant polypeptides are made using methods known in the art such as oligonucleotide-mediated (site-directed) mutagenesis, alanine scanning, and PCR mutagenesis. Site direct mutagenesis (Carter, 1986; Zoller and Smith, 1987), cassette mutagenesis, restriction selection mutagenesis (Wells et al., 1985) or other known techniques can be performed on the cloned DNA to produce CD16 variants (Ausubel, 2002; Sambrook and Russell. 2001).

Optionally, the CAR targets an antigen associated with a specific cancer type. Optionally, the cancer is selected from the group consisting of leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia), polycythemia vera, lymphomas (e.g., Hodgkin's disease and non-Hodgkin's disease), multiple myeloma, Waldenstrom's macroglobulinemia, heavy chain disease, solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

In some embodiments, a polynucleotide encoding a CAR is mutated to alter the amino acid sequence encoding for CAR without altering the function of the CAR. For example, polynucleotide substitutions leading to amino acid substitutions at “non-essential” amino acid residues can be made in the CARs disclosed above. CARs can be engineered as described, for example, in Patent Publication Nos. WO 2014039523; US 20140242701; US 20140274909; US 20130280285; and WO 2014099671, each of which is incorporated herein by reference in its entirety. Optionally, the CAR is a CD19 CAR, a CD33 CAR or CSPG-4 CAR.

### Additional Modifications—Cytokines

The cytotoxicity of NK-92™ cells is dependent on the presence of cytokines (e.g., interleukin-2 (IL-2). The cost of using exogenously added IL-2 needed to maintain and expand NK-92™ cells in commercial scale culture is significant. The administration of IL-2 to human subjects in sufficient quantity to continue activation of NK92 cells would cause adverse side effects.

In some embodiments, FcR-expressing NK-92® cells are further modified to express at least one cytokine and a suicide gene. In specific embodiments, the at least one cytokine is IL-2, IL-12, IL-15, IL-18, IL-21 or a variant thereof. In preferred embodiments, the cytokine is IL-2. In certain embodiments the IL-2 is a variant that is targeted to the endoplasmic reticulum (“ER”). Exemplary sequences for IL-2 and IL-2 variants that are targeted to the ER are disclosed in U.S. Pat. Pub. No. 20180193383A1, the relevant disclosure is herein incorporated by reference.

In one embodiment, the suicide gene is the thymidine kinase (TK) gene. The TK gene may be a wild-type or mutant TK gene (e.g., tk30, tk75, sr39tk). Cells expressing the TK protein can be killed using ganciclovir.

In another embodiment, the suicide gene is Cytosine deaminase which is toxic to cells in the presence of 5-fluorocytosine. Garcia-Sanchez et al. “Cytosine deaminase adenoviral vector and 5-fluorocytosine selectively reduce breast cancer cells 1 million-fold when they contaminate hematopoietic cells: a potential purging method for autologous transplantation.” Blood 1998 Jul. 15; 92(2):672-82.

In another embodiment, the suicide gene is cytochrome P450 which is toxic in the presence of ifosfamide, or cyclophosphamide. See e.g., Touati et al. “A suicide gene therapy combining the improvement of cyclophosphamide tumor cytotoxicity and the development of an anti-tumor immune response.” Curr Gene Ther. 2014; 14(3):236-46.

In another embodiment, the suicide gene is iCas9. Di Stasi, (2011) “Inducible apoptosis as a safety switch for adoptive cell therapy.” N Engl J Med 365: 1673-1683. See also Morgan, “Live and Let Die: New Suicide Gene Therapy Moves to the Clinic” Molecular Therapy (2012); 20: 11-13. The iCas9 protein induces apoptosis in the presence of a small molecule AP1903. AP1903 is biologically inert small molecule, that has been shown in clinical studies to be well tolerated, and has been used in the context of adoptive cell therapy.

In one embodiment, the modified NK-92® cells are irradiated prior to administration to the patient. Irradiation of NK-92® cells is described, for example, in U.S. Pat. No. 8,034,332, which is incorporated herein by reference in its entirety. In one embodiment, modified NK-92® cells that have not been engineered to express a suicide gene are irradiated.

### Transgene Expression

Transgenes (e.g., CD19 CAR and CD16) can be engineered into an expression vector by any mechanism known to those of skill in the art. Transgenes may be engineered into the same expression vector or a different expression vector. In preferred embodiments, the transgenes are engineered into the same vector.

In some embodiments, the vector allows incorporation of the transgenes) into the genome of the cell. In some embodiments, the vectors have a positive selection marker. Positive selection markers include any genes that allow the cell to grow under conditions that would kill a cell not expressing the gene. Non-limiting examples include antibiotic resistance, e.g., geneticin (Neo gene from Tn5).

Any number of vectors can be used to express the Fc receptor and/or the CAR. In some embodiments, the vector is a plasmid. In one embodiment, the vector is a viral vector. Viral vectors include, but are not limited to, retroviral vectors, adenoviral vectors, adeno-associated viral vectors, herpes simplex viral vectors, pox viral vectors, and others.

Transgenes can be introduced into the NK-92™ cells using any transfection method known in the art, including, by way of non-limiting example, infection, electroporation, lipofection, nucleofection, or “gene-gun.”

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### Embodiments

This disclosure comprises the following, non-limiting embodiments:

Embodiment 1. A method of freezing NK-92®cells, the method comprising: contacting NK-92® cells with a cell freezing medium, wherein the cell freezing medium comprises a first composition and a second composition, wherein the first composition comprises: (a) one or more electrolytes selected from the group consisting of potassium ions, sodium ions, magnesium ions, and calcium ions; (b) one or more macromolecular agents selected from the group consisting of human albumin, polysaccharide and colloidal starch; (c) a pH buffer; (d) at least one sugar; (e) at least one sugar alcohol; (f) an impermeant agent selected from the group consisting of lactobionate, gluconate, citrate and glycerophosphate; and (g) DMSO; and wherein the second composition comprises albumin.

Embodiment 1. The method of embodiment 1, wherein the NK-92® cells are contacted with the second composition before adding the first composition.

Embodiment 2. The method of embodiment 1, wherein the sugar alcohol is a 6-carbon sugar alcohol.

Embodiment 3. The method of embodiment 1, wherein the sugar is a simple sugar.

Embodiment 4. The method of embodiment 1, wherein the simple sugar is selected from the group consisting of glucose, dextran, dextrose, trehalose, and maltose.

Embodiment 5. The method of embodiment 1, wherein the albumin in the second composition is human albumin.

Embodiment 6. The method of embodiment 1, wherein the second composition comprises 2-10% human albumin.

Embodiment 7. The method of any of embodiments 1-7, wherein the first and second composition are mixed at a volume ratio that ranges from 1:5 to 5:1.

Embodiment 8. The method of embodiment 1, wherein first composition comprises 10% DMSO.

Embodiment 9. The method of embodiment 1, wherein the first composition further comprises one or more substrates effective for regeneration of ATP.

Embodiment 10. The method of embodiment 10, wherein the one or more substrates are selected from the group consisting of adenosine, fructose, ribose and adenine.

Embodiment 11. The method of any of embodiments 1-11, wherein the first composition further comprises glutathione.

Embodiment 12. The method of any of embodiments 1-12, wherein the first composition further comprises one or more impermeant agents that can maintain ionic and osmotic balance during hypothermia.

Embodiment 13. The method of any of embodiments 1-13, wherein the first composition and second composition is mixed at a volume ratio between 1:5 and 5:1.

Embodiment 14. The method of any of embodiments 1-13, wherein a cell freezing medium comprises less than 10% DMSO.

Embodiment 15. The method of any of embodiments 1-13, wherein the method further comprises freezing the cells using a controlled rate freeze to reach a final temperature of −80° C. or lower.

Embodiment 16. The method of any of embodiments 1-16, wherein the method further comprises storing the cells at −80° C. to −196° C.

Embodiment 17. The method of any of embodiments 16-17, wherein the method further comprises thawing the preserved cells.

Embodiment 18. The method of embodiment 18, wherein the cells are thawed at 37° C.

Embodiment 19. The method of embodiment 9, wherein more than 70% of the cells are viable at the time of thawing.

Embodiment 20. The method of embodiment 9, wherein more than 90% of the cells are viable at the time of thawing.

Embodiment 21. The method of embodiment 18, wherein the thawed cells have a direct cytoxicity of at least 80% against K562 cells at an effector to target ratio of 10:1.

Embodiment 22. The method of embodiment 18, wherein the thawed cells have a direct cytotoxicity that is 70-100% of that of the NK-92® cells before the cells are frozen.

Embodiment 23. The method of embodiment 18, wherein the thawed NK-92® cells have a ADCC activity of at least 80-120% against Ramos cells at an effector to target ratio of 10:1, in the presence of an antibody targeting the Ramos cells.

Embodiment 24. The method of embodiment 18, wherein the thawed NK-92® cells have a ADCC activity that is 80-100% of that of the cells before the cells are frozen.

Embodiment 25. The method of embodiment 19, wherein the method further comprises administering the thawed cells to a patient in need via infusion.

Embodiment 26. The method of embodiment 1, wherein the NK-92® cells express a cytokine, Fc Receptor, chimeric antigen receptor, or a combination thereof.

Embodiment 27. A NK-92® cell culture comprising: NK-92® cells and a cell freezing medium, wherein the cell freezing medium comprises a first composition and a second composition, wherein the first composition comprises: (a) one or more electrolytes selected from the group consisting of potassium ions, sodium ions, magnesium ions, and calcium ions; (b) one or more macromolecular agents selected from the group consisting of human albumin, polysaccharide and colloidal starch; (c) a pH buffer; (d) at least one sugar; (e) at least one sugar alcohol; (f) an impermeant agent being at least one member selected from the group consisting of lactobionate, gluconate, citrate and glycerophosphate; and (g) DMSO; and wherein the second composition comprises albumin.

Embodiment 28. The NK-92® cell culture of embodiment 28, wherein the sugar alcohol is a 6-carbon sugar alcohol.

Embodiment 29. The NK-92® cell culture of embodiment 28, wherein the sugar is a simple sugar.

Embodiment 30. The NK-92® cell culture of embodiment 30, wherein the simple sugar is selected from the group consisting of glucose, dextran, dextrose, trehalose, and maltose.

Embodiment 31. The NK-92® cell culture of embodiment 28, wherein the second composition is human albumin.

Embodiment 32. The NK-92® cell culture of embodiment 32, wherein the second composition is 2-10% human albumin.

Embodiment 33. The NK-92® cell culture of any of embodiments 28-33, wherein the first composition and second composition is mixed at a volume ratio ranging from 5:1 to 1:5.

Embodiment 34. The NK-92® cell culture of embodiment 28, wherein the first composition comprises 10% DMSO.

Embodiment 35. The NK-92® cell culture of embodiment 28, wherein the first composition further comprises a substrate effective for the regeneration of ATP.

Embodiment 36. The NK-92® cell culture of embodiment 36, wherein the substrate in the first composition is selected from the group consisting of adenosine, fructose, ribose and adenine.

Embodiment 37. The NK-92® cell culture of embodiment 28, wherein the first composition comprises 20-60 mM potassium ions.

Embodiment 38. The NK-92® cell culture of embodiment 28, wherein the first composition comprises 50-150 mM sodium ions.

Embodiment 39. The NK-92® cell culture of embodiment 28, wherein the first composition comprises 0.001-1 mM magnesium ions.

Embodiment 40. The NK-92® cell culture of any of embodiments 28-40, wherein the first composition further comprises glutathione.

Embodiment 41. The NK-92® cell culture of embodiment 28, wherein the cell culture is kept at 80° C. to −196° C.

Embodiment 42. The NK-92® cell culture of embodiment 42, wherein at least 70% of NK-92™ cells in the cell culture that has been kept at −80° C. to −196° C. are viable after cell culture is thawed.

Embodiment 43. The NK-92® cell culture of any of embodiments 28-43, wherein the NK-92™ cells comprise a cytokine, Fc Receptor, chimeric antigen receptor, or a combination thereof.

Embodiment 44. A cell freezing medium produced by mixing a first composition and a second composition at a one to one ratio, wherein the first composition comprises: (a) one or more electrolytes selected from the group consisting of potassium ions, sodium ions, magnesium ions, and calcium ions; (b) one or more macromolecular agents selected from the group consisting of human albumin, polysaccharide and colloidal starch; (c) a pH buffer; (d) at least one sugar; (e) at least one sugar alcohol; (f) an impermeant agent selected from the group consisting of lactobionate, gluconate, citrate and glycerophosphate; and (g) DMSO; and wherein the second composition comprises albumin.

### EXAMPLES

The following examples are for illustrative purposes only and should not be interpreted as limitations. There are a variety of alternative techniques and procedures available to those of skill in the art which would similarly permit one to successfully perform the examples below.

### Example 1: NK-92® Cell Formulation Freeze and Thaw Procedures

NK-92® cells were expanded in Xuri bioreactors, harvested and concentrated using kSep400 centrifuge. Concentrated cells were resuspended in the cell freezing medium, which is a mixture of 5% HA and CryoStor®10 (CS10), which comprises the components as listed in Table 1, at a volume ratio of 1:1, for cryopreservation. Formulated cells are transferred to the Cell Freeze® cryobags (CF-750 supplied by Charter Medical Limited, Winston-Salem, N.C.) at the proposed clinical dose (2×10e7 cells/mL, 100 mL volume). Cell Freeze® cryobags were subsequently frozen using a CryoMed controlled rate freezer using a preset freezing Profile 4. Frozen bags were stored in vapor phase of a LN2 cryofreezer (≤120° C.). Frozen cells were removed from the LN2 freezer when ready to thaw for infusion. Cryofreeze bags were transferred immediately to a water bath containing a beaker with water at 37° C.

### Example 2: Viability of the NK-92® Cells After Thawing

After thaw, the cells from each bag/vial were counted and the total viable cells collected was calculated. High cell viability (>90%) was observed at thaw. Viability of the cells were determined and shown in the Table 4 below. A fresh NK-92® cell sample in the same cell freezing medium before freezing was used as control (“Pre-freeze”) and counted and viability checked.

TABLE 4
Viability Data

| | % Viability at | % Viability |
|---|---|---|
| Experiment | Pre-freeze | at thaw |
| #1 | 96.3% | 91.2% |
| #2 | 96.8% | 95.2% |
| #3 | 94% | 90% |
| #4 | 95.3% | 91% |
| #5 | 96.5% | 95.1% |
| #6 | 95.9% | 95.3% |

Note that the NK-92 ® cells used in Example 2 are aNK ™ cells.

### Example 3: Phenotypes of the NK-92® Cells After Thawing

A study was conducted to quantify the expression of a panel of six surface protein markers on the NK-92® cells that has been thawed as described in Example 1. The expression profile is compared to the profile of the NK-92™ cells in the control sample. The panel of surface markers was selected to be representative of natural killer (NK) cells. The surface markers CD54, CD56, NKG2D, NKp30, CD3, and CD16 were analyzed and the marker expression was determined by staining cells with specific fluorochrome-conjugated antibodies and detecting bound antibodies by flow cytometry.

The results show that expression of surface markers (CD56, CD54, CD3, CD16, NKG2D and NKp30) on NK-92® cells (aNK™ cells or haNK® cells) was comparable between the control samples (fresh samples) and those that had been cryopreserved using CryoStor®10 (CS10) +HA Surface marker expression on NK-92® cells were analyzed by flow cytometry-based staining and the results are presented in Table 5 below.

TABLE 5

| | Phenotypes of Cryopreserved NK-92 ™ cells at thaw | | | | | | |
|---|---|---|---|---|---|---|---|
| | Description | CD3 | CD56 | CD16 | CD54 | NKG2D | NKp30 |
| Experi- | aNK ™ cells at | 0.90 | 99.90 | 4.50 | 99.70 | 98.50 | 98.50 |
| ment #1 | Pre-freeze | | | | | | |
| | aNK ™ cells | 0.30 | 99.70 | 1.30 | 99.80 | 99.00 | 95.80 |
| | post thaw | | | | | | |
| Experi- | haNK ® cells at | 0.11 | 99.98 | 98.82 | 99.95 | 83.03 | 99.16 |
| ment #2 | Pre-freeze | | | | | | |
| | haNK ® cells | 0.34 | 99.88 | 98.68 | 99.94 | 80.32 | 99.66 |
| | post thaw | | | | | | |
| Experi- | haNK ® cells at | 0.07 | 99.7 | 97.73 | 99.82 | 90.41 | 99.39 |
| ment # | Pre-freeze | | | | | | |
| | haNK ® cells | 0.28 | 99.82 | 98.57 | 99.91 | 89.48 | 99.83 |
| | post thaw | | | | | | |

### Example 4: Cytotoxicity of the NK-92® Cells After Thawing

Direct cytotoxicity of the thawed aNK™ cells after cryopreservation was evaluated against K562 cell line. aNK™ cells were mixed with the calcein loaded target cells at the Effector:Target ratio of 10:1. Calcein release was assessed by fluorescence plate reader post 3 hours of co-incubation. The results are shown in Table 6 below.

TABLE 6
Direct Cytotoxicity

| Description | % Cytotoxicity |
|---|---|
| aNK ™ at | 97 ± 5 |
| Pre-freeze | |
| aNK ™ | 88 ± 1 |
| at thaw | |

Comparison of the control samples with cryopreserved test samples showed that cryopreserved NK-92™ cells retained their direct cytotoxicity of K562 target cells at thaw; the percent of direct cytotoxicity was shown as 88±1%.

haNK® cells were mixed with the calcein labeled Ramos cells (target cells) in the presence of Rituxan antibody, which targets the CD20 expressed on target cells, at the Effector:Target ratio of 10:1. The anti β-gal antibody was used as a control for cytotoxicity not related to ADCC. Calcein release was assessed by fluorescence plate reader post 3 hour of co-incubation. Cytotoxicity was determined using a Calcein-release assay and is expressed as the percentage (%) of Calcein release at an Effector:Target ratio of 10:1 (mean±standard deviation). Each sample was assayed in triplicate. The results are shown in Table 7.

TABLE 7
ADCC

| | | % Specific lysis | |
|---|---|---|---|
| Experiment | Sample | Rituxan | Anti β-gal |
| #1 | Pre-Freeze | 107 ± 4 | 13 ± 1 |
| | Post-Thaw | 99 ± 2 | 12 ± 2 |
| #2 | Pre-Freeze | 106 ± 5 | 16 ± 1 |
| | Post-Thaw | 95 ± 4 | 14 ± 1 |

The results show that the cells that have been frozen and thawed (post-thaw state) as described retains ADCC activity, e.g., 99±2% or 95±4%. And the ADCC activity of the cells at post-thaw state is substantially the same as, e.g., about 92.5% (99/107), or 90% (95/106) of the ADCC activity of the cells at the pre-freeze state.

Informal Sequence Listing

High Affinity Variant Immunoglobulin Gamma Fe Region Receptor IMA
nucleic acid sequence (full length form).
SEQ ID NO: 1
ATGTGGCA GCTGCTGCTG CCTACAGCTC TCCTGCTGCT GGTGTCCGCC
GGCATGAGAA CCGAGGATCT GCCTAAGGCC GTGGTGTTCC TGGAACCCCA
GTGGTACAGA GTGCTGGAAA AGGACAGCGT GACCCTGAAG TGCCAGGGCG
CCTACAGCCC CGAGGACAAT AGCACCCAGT GGTTCCACAA CGAGAGCCTG
ATCAGCAGCC AGGCCAGCAG CIACITCATCGACGCCGCCA CCGTGGACGA
CAGCGGCGAG TATAGATGCC AGACCAACCT GAGCACCCTGAGCGACCCCG
TGCAGCTGGA AGTGCACATC GGATGGCTGC TGCTGCAGGC
CCCCAGATGGGTGTTCAAAG AAGAGGACCC CATCCACCTG AGATGCCACT
CTTGGAAGAA CACCGCCCTGCACAAAGTGA CCTACCTGCA GAACGGCAAG
GGCAGAAAGT ACTTCCACCA CAACAGCGAC TTCTACATCC CCAAGGCCAC
CCTGAAGGAC TCCGGCTCCT ACTTCTGCAG AGGCCTCGTGGGCAGCAAGA
ACGTGTCCAG CGAGACAGTG AACATCACCA TCACCCAGGG
CCTGGCCGTGTCTACCATCA GCAGCTTTTT CCCACCCGGC TACCAGGTGT
CCTTCTGCCT CGTGATGGTGCTGCTGTTCG CCGTGGACAC CGGCCTGTAC
TTCAGCGTGA AAACAAACAT CAGAAGCAGCACCCGGGACT GGAAGGACCA
CAAGTTCAAG TGGCGGAAGG ACCCCCAGGA CAAGTGA
High Affinity Variant Immunoglobulin Gamma Fe Region Receptor III-A
amino acid sequence (full length form). The Val at position 176 is underlined.
SEQ ID NO: 2
Met Trp Gln Leu Leu Leu Pro Thr Ala Leu Leu Leu Leu Val Ser Ala Gly Met Arg Thr Glu Asp
Leu Pro Lys Ala Val Val Phe Leu Glu Pro Gln Trp Tyr Arg Val Leu Glu Lys Asp Ser Val Thr
Leu Lys Cys Gln Gly Ala Tyr Ser Pro Glu Asp Asn Ser Thr Gln Trp Phe His Asn Glu Ser Leu
Ile Ser Ser Gln Ala Ser Ser Tyr Phe Ile Asp Ala Ala Thr Val Asp Asp Ser Gly Glu Tyr Arg Cys
Gln Thr Asn Leu Ser Thr Leu Ser Asp Pro Val Gln Leu Glu Val His Ile Gly Trp Leu Leu Leu
Gln Ala Pro Arg Trp Val Phe Lys Glu Glu Asp Pro Ile His Leu Arg Cys His Ser Trp Lys Asn
Thr Ala Leu His Lys Val Thr Tyr Leu Gln Asn Gly Lys Gly Arg Lys Tyr Phe His His Asn Ser
Asp Phe Tyr Ile Pro Lys Ala Thr Leu Lys Asp Ser Gly Ser Tyr Phe Cys Arg Gly Leu Val Gly
Ser Lys Asn Val Ser Sec Glu Thr Val Asn Ile Thr Ile Thr Gln Gly Leu Ala Val Ser Thr Ile Ser
Sec Phe Phe Pro Pro Gly Tyr Gln Val Ser Phe Cys Leu Val Met Val Leu Leu Phe Ala Val Asp
Thr Gly Leu Tyr Phe Ser Val Lys Thr Asn Ile Arg Ser Ser Thr Arg Asp Trp Lys Asp His Lys
Phe Lys Trp Arg Lys Asp Pro Gln Asp Lys

1. A method of freezing NK-92®cells, the method comprising:
contacting NK-92® cells with a cell freezing medium, wherein the cell freezing medium comprises a first composition and a second composition, wherein the first composition comprises:
(a) one or more electrolytes selected from the group consisting of potassium ions, sodium ions, magnesium ions, and calcium ions;
(b) one or more macromolecular agents selected from the group consisting of human albumin, polysaccharide and colloidal starch;
(c) a pH buffer;
(d) at least one sugar;
(e) at least one sugar alcohol;
(f) an impermeant agent selected from the group consisting of lactobionate, gluconate, citrate and glycerophosphate; and
(g) DMSO;
and wherein the second composition comprises albumin.

2. The method of claim 1, wherein the NK-92® cells are contacted with the second composition before adding the first composition.

3. The method of claim 1, wherein the sugar alcohol is a 6-carbon sugar alcohol.

4. The method of claim 1, wherein the sugar is a simple sugar.

5. (canceled)

6. The method of claim 1, wherein the albumin in the second composition is human albumin.

7-8. (canceled)

9. The method of claim 1, wherein first composition comprises one or more of the following: 10% DMSO, one or more substrates effective for regeneration of ATP, one or more impermeant agents that can maintain ionic and osmotic balance during hypothermia, and glutathione.

10. (canceled)

11. The method of claim 10, wherein the one or more substrates are selected from the group consisting of adenosine, fructose, ribose and adenine.

12-13. (canceled)

14. The method of claim 1, wherein the first composition and second composition is mixed at a volume ratio between 1:5 and 5:1.

15. The method of claim 1, wherein a cell freezing medium comprises less than 10% DMSO.

16. The method of claim 1 , wherein the method further comprises freezing the cells using a controlled rate freeze to reach a final temperature of −80° C. or lower.

17. The method of claim 1, wherein the method further comprises storing the cells at −80° C. to −196° C.

18. The method of claim 16, wherein the method further comprises thawing the preserved cells and administering the thawed cells to a patient in need via infusion.

19. (canceled)

20. The method of claim 9, wherein more than 70% of the cells are viable at the time of thawing.

21. (canceled)

22. The method of claim 18, wherein the thawed cells
i) have a direct cytoxicity of at least 80% against K562 cells at an effector to target ratio of 10:1;
ii) have a direct cytotoxicity that is 70-100% of that of the NK-92® cells before the cells are frozen;
iii) have a ADCC activity of at least 80-120% against Ramos cells at an effector to target ratio of 10:1, in the presence of an antibody targeting the Ramos cells; or
iv) have a ADCC activity that is 80-100% of that of the cells before the cells are frozen.

23-26. (canceled)

27. The method of claim 1, wherein the NK-92® cells express a cytokine, Fc Receptor, chimeric antigen receptor, or any combination thereof.

28. A NK-92® cell culture comprising:
NK-92® cells and a cell freezing medium, wherein the cell freezing medium comprises a first composition and a second composition, wherein the first composition comprises:
(a) one or more electrolytes selected from the group consisting of potassium ions, sodium ions, magnesium ions, and calcium ions;
(b) one or more macromolecular agents selected from the group consisting of human albumin, polysaccharide and colloidal starch;
(c) a pH buffer;
(d) at least one sugar;
(e) at least one sugar alcohol;
(f) an impermeant agent being at least one member selected from the group consisting of lactobionate, gluconate, citrate and glycerophosphate; and
(g) DMSO; and
wherein the second composition comprises albumin.

29. The NK-92® cell culture of claim 28, wherein the sugar alcohol is a 6-carbon sugar alcohol.

30. The NK-92® cell culture of claim 28, wherein the sugar is a simple sugar.

31. (canceled)

32. The NK-92® cell culture of claim 28, wherein the second composition is human albumin.

33. (canceled)

34. The NK-92® cell culture of claim 28, wherein the first composition and second composition is mixed at a volume ratio ranging from 5:1 to 1:5.

35. The NK-92® cell culture of claim 28, wherein the first composition comprises one or more of the following: 10% DMSO, a substrate effective for the regeneration of ATP, 20-60 mM potassium ions, 50-150 mM sodium ions, 0.001-1 mM magnesium ions, glutathione.

36. (canceled)

37. The NK-92® cell culture of claim 36, wherein the substrate in the first composition is selected from the group consisting of adenosine, fructose, ribose and adenine.

38-41. (canceled)

42. The NK-92® cell culture of claim 28, wherein the cell culture is kept at 80° C. to −196° C.

43. The NK-92® cell culture of claim 42, wherein at least 70% of NK-92® cells in the cell culture that has been kept at −80° C. to −196° C. are viable after cell culture is thawed.

44. The NK-92® cell culture of claim 28, wherein the NK-92® cells comprise a cytokine, Fc Receptor, chimeric antigen receptor, or a combination thereof.

45. A cell freezing medium produced by mixing a first composition and a second composition at a one to one ratio, wherein the first composition comprises:
(a) one or more electrolytes selected from the group consisting of potassium ions, sodium ions, magnesium ions, and calcium ions;
(b) one or more macromolecular agents selected from the group consisting of human albumin, polysaccharide and colloidal starch;
(c) a pH buffer;
(d) at least one sugar;
(e) at least one sugar alcohol;
(f) an impermeant agent selected from the group consisting of lactobionate, gluconate, citrate and glycerophosphate; and
(g) DMSO;
and wherein the second composition comprises albumin.


SEQUENCE LISTING (2 sequences)

SEQ ID NO: 1 - DNA, 765 bp, Artificial Sequence
  source: /note=“Description of Artificial Sequence Synthetic polynucleotide”
atgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggctaccaggtgtccttctgcctcgtgatggtgctgctgttcgccgtggacaccggcctgtacttcagcgtgaaaacaaacatcagaagcagcacccgggactggaaggaccacaagttcaagtggcggaaggacccccaggacaagtga

SEQ ID NO: 2 - protein, 254 aa, Artificial Sequence
  source: /note=“Description of Artificial Sequence Synthetic polypeptide”
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK